(19) United States
(12) Patent Application Publication
(10) Pub. No.: US 2007/0021644 A1
(43) Pub. Date: Jan. 25, 2007

(54) NONINVASIVE METHODS, APPARATUS, KITS, AND SYSTEMS FOR INTRAOPERATIVE POSITION AND LENGTH DETERMINATION

(76) Inventors: Steven T. Woolson, Palo Alto, CA (US); John C. Radke, Whitefish Bay, WI (US)

Correspondence Address: WILSON SONSINI GOODRICH & ROSATI, 650 PAGE MILL ROAD, PALO ALTO, CA, 94304-1050, US

(21) Appl. No.: 11/276,467

(22) Filed: Mar. 1, 2006

Related U.S. Application Data
(60) Provisional application No. 60/658,100, filed on Mar. 2, 2005.

(51) Int. Cl. A61N 2/00

(52) U.S. Cl. 600/9

(57) ABSTRACT

Methods, apparatus, kits and systems are presented for determining the intraoperative position of at least a first anatomic location of a surgical patient and optionally for measuring the distance between at least first and second anatomic locations of a surgical patient.

### CROSS-REFERENCE

This application claims the benefit of U.S. Provisional Application No. 60/658,100, filed Mar. 2, 2005, which is incorporated herein by reference in its entirety.

### BACKGROUND OF THE INVENTION

Total hip replacement surgery (THR), also known as total hip arthroplasty, was introduced into clinical practice over thirty years ago and is now performed nearly 250,000 times a year in the United States to relieve pain and improve function in patients who have severe arthritis of the hip joint.

The majority of patients undergoing THR enter surgery with unequal leg lengths, with the affected leg shorter than the normal leg due to loss of the hip joint articular cartilage and erosion of bone from the femoral head due to the arthritic process. Differences in leg length affects gait, may cause instability of the hip joint, and may exacerbate pain. The usual preoperative leg length discrepancy in patients undergoing THR ranges between 4 and 10 mm, but on rare occasions can reach as much as 2.5 cm. Total hip arthroplasty can easily equalize the majority of such leg length differences, since the implantation of hip components that are longer than the resected femoral head and neck can lengthen the leg up to 1-2 cm. The maintenance or restoration of equal leg lengths is thus an important therapeutic goal of THR surgery.

In addition, the surgeon's failure to equalize or maintain leg length during THR is readily noticeable, and is a frequent source of patient dissatisfaction with THR surgery.

The methods and apparatus currently available to measure leg length during THR surgery, however, are typically inaccurate, often invasive, and at times complex.

Simple, direct comparison of leg lengths by visually comparing the positions of the medial malleoli of the ankles is not a valid measurement when patients are in the lateral decubitus position, the position used by almost all surgeons, since the pelvis is oblique and not level in this position. The “shuck” test, in which the surgeon pulls longitudinally on the leg to check how far the joint may be distracted with a trial implant in place, merely assesses the tightness of the soft tissues around the hip joint and is often inaccurate for assessing leg length. Radiographic assessment of leg lengths is helpful only if the patient is in the supine position, a rarely used surgical approach.

Invasive methods of leg length comparison and equalization are typically based on the direct physical measurement of the distance between first and second reference points fixedly attached to the pelvis and femur. Usually, the pelvic reference point consists of a metal pin that is drilled or driven into the wing of the ilium by the surgeon; the femoral reference point is usually a second pin inserted into the femur or a mark made on the lateral aspect of the greater trochanter or proximal femur with electrocautery.

Various mechanical devices, of varying complexity, are used to measure the distance between the two reference points. See, e.g., U.S. Pat. Nos. 5,122,145, 5,318,571; 5,700,268; 5,755,794; 5,788,705; 5,814,050; 6,027,507; 6,632,226; 6,645,214; and international patent application publication WO 01/30247. U.S. Pat. Nos. 6,383,149 and 6,685,655 disclose the analogous use of a handheld device having two laser sources disposed a fixed distance from one another to measure the distance between reference points surgically affixed to the pelvis and femur.

Whether performed by mechanical device or by laser, the measurement between the pelvic pin and femoral reference point is frequently inaccurate, in part due to the difficulty of fixedly securing the pin to the ilium for the duration of the procedure. The wing of the ilium is thin (typically 1 cm or less) and the pelvic pin is easily loosened during the surgical procedure despite care taken to avoid accidental contact. In addition, the distance can be affected by the relative angular position of the femur within the acetabulum.

In light of these known inaccuracies, other, more complex, computerized approaches have been suggested; none is used in typical surgical practice.

U.S. patent application publication no. 2004/0230199 describes a computer assisted system for hip replacement surgery. Markers that are optically trackable in space—such as retro-reflective spheres—are secured and anchored to the pelvis and the femur. Computerized tracking of the markers, combined with other digitized data, such as digitized bone topographic data, are used to calculate a desired implant position for the femoral implant as a function of the limb length; computer guidance is provided to the surgeon to assist in altering the femur.

U.S. Pat. No. 6,711,431 similarly discloses a computer assisted optical tracking navigation system for hip replacement surgery. The visible markers are attached to bone intraoperatively, preferably by use of a ligature that obviates the use of bone screw, pins, or other bone damaging means.

U.S. patent application publication no. 2003/0105470 discloses an electromagnetic telemetry-based position monitoring system for determining relative bone positions and leg length. At least one, typically two, telemetry transmitters are attached to the patient. In the presence of a magnetic field created by an external field generator, the devices actively transmit their position and orientation via wired or wireless communication links to a processing device. In certain of the disclosed embodiments, the telemetry transmitter is attached adhesively to the skin of patient.

Each of the above-described approaches suffers from one or more of inaccuracy, invasiveness, and mechanical and/or computational complexity. There thus exists a continuing need in the art for precise, noninvasive, simple methods, systems, and apparatus for determining the absolute and relative position of bones, including limbs, during surgery. There exists in particular a need in the art for precise, noninvasive, and simple methods, systems, and apparatus for determining leg length during hip surgeries, such as total hip replacement, repair of hip fracture, osteotomies, and pediatric reconstructive procedures.

### SUMMARY OF THE INVENTION

The present invention solves these and other needs in the art by using magnetic field sources, typically permanent magnets, to provide simple, inexpensive, disposable markers for desired anatomic locations; the location of the magnetic markers is readily sensed, without dedicated communication links, using magnetic field sensing apparatus positioned external to the patient.

The markers can be applied noninvasively, outside the sterile surgical field, to the skin overlying readily identifiable anatomic landmarks. In hip arthroplasty, for example, the markers can be applied with adhesive to the skin overlying the lateral iliac crest and the fibular head. In alternative embodiments, sterilized markers can be applied inside the surgical field, even to bone or soft tissue exposed during surgery.

In exemplary embodiments, a simple, handheld, battery-operated, magnetic sensor unit can then be used to determine and compare the preoperative and intraoperative location of the magnets; in other embodiments, a more elaborate computerized system can be used to track the position of the markers in three dimensions over time.

Using the methods, systems, and apparatus of the present invention during total hip replacement surgery, the leg length achieved intraoperatively upon implantation of one or more trial prostheses can readily be compared to the preoperative leg length, thus facilitating selection of a prosthesis that best equalizes the length of the involved and uninvolved legs.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The above and other objects and advantages of the present invention will be apparent upon consideration of the following detailed description taken in conjunction with the accompanying drawings, in which like characters refer to like parts throughout, and in which:

FIG. 1 is a posterior view of the normal right hip, including the pelvis superiorly to the iliac crest and the femur inferiorly to the knee, with the head of the femur anatomically engaged in the acetabulum;

FIG. 2A is a posterior view of a patient in lateral decubitus position as positioned for total hip replacement surgery, with the pelvis indicated in outline, further illustrating lateral prominences marked externally with magnetic field sources A and B according to an embodiment of the methods of the present invention; surgical drapes are omitted and the center of the hip joint is marked as “C”;

FIG. 2B is a top view of the same patient, further illustrating in schematic an embodiment of a magnetic sensor device of the present invention positioned posteriorly for measuring the distance between external magnetic markers A and B, according to the methods of the present invention;

FIG. 2C is a posterior view of the right hip showing three exemplary magnetic field sources reversibly fixed to skin overlying the ilium, the greater trochanter of the femur, and the lateral condyle of the tibial head, with two measurements, D1 and D2, that may usefully be made intraoperatively according to the present invention;

FIG. 3A is a top perspective view of an exemplary embodiment of a hand held magnetic sensor for intraoperative use, according to the present invention;

FIG. 3B is a partial bottom perspective view of the embodiment shown in FIG. 3A; and

FIG. 4 is a plan view of another exemplary embodiment of a hand held magnetic sensor for intraoperative use, according to the present invention.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention applies magnetic sensing technology to monitor the spatial position of desired anatomic locations and to determine the distance between two or more such anatomic locations during surgery.

In a first aspect, the invention provides methods for determining the spatial position of at least one anatomic location of a patient during surgery. The method comprises intraoperatively detecting the position of a first magnetic field source reversibly fixed to a first desired anatomic location of a patient. In some embodiments, the magnetic field source is an electromagnet. In other embodiments, the field source is a permanent magnet.

Using magnetic field sources as anatomic markers obviates the requirement for a direct line of sight between the marker and external sensing device, as is required by optical markers; using permanent magnets as field sources obviates the need for a power source running to the patient and the need for embedded circuitry or electronic signaling means within the anatomic marker, permitting the markers in certain embodiments to be inexpensive, disposable, and in some embodiments, readily sterilizable.

The magnet can be of any composition known for manufacture of permanent magnets; the magnet can be selected, for example, from the group consisting of neodymium-iron-boron (Nd—Fe—B) magnets (sintered or bonded), samarium-cobalt (Sm—Co) magnets, alnico magnets, and ferrite (ceramic) magnets.

The magnet can be formed in any convenient shape, such as a round, a cylinder, a square, a block, a ring, an arc, or a wafer, with certain embodiments usefully being nonsymmetric. The magnet can be rigid or, in certain embodiments, can be formed from a flexible magnetic sheet; such flexible sheet magnets typically combine ceramic ferrite magnet powder with a flexible thermoplastic binder. Such flexible magnets can readily be molded into desired shapes that may usefully be wedged, folded, or wrapped around a desired anatomic location during surgery.

The magnet can conveniently be sized for ease of manual application to the skin surface, with maximum dimension typically less than about 5 cm, often with maximum dimension less than about 4.5 cm, 4.0 cm, 3.5 cm, 3 cm, even with maximum dimension less than about 2.5, 2.4, 2.3, 2.2, even as little as 2.1 or 2 cm, and even less. In certain embodiments formed other than as a sheet, the magnet can be at least about 0.5 cm in smallest dimension, in typical embodiments at least about 0.6, 0.7, 0.8, 0.9, even at least about 1.0, 1.1, 1.2, 1.3, 1.4, or 1.5 cm or more in smallest dimension.

In typical embodiments, the magnet can be inexpensive and disposable. The magnet can be used unsealed, or, in alternative embodiments, can be sealed, either porously or hermetically. Magnets suitable for use in the methods of the present invention are available commercially from a wide variety of suppliers, such as Dexter Magnetic Technologies (Fremont, Calif.); Magnet City (Sunrise, Fla.); and Magnetic Component Engineering, Inc (Torrance, Calif.).

In some embodiments, the method further comprises detecting the position of a second magnetic field source reversibly fixed to a second desired anatomic location of a patient. As with the first magnetic field source, the second source may be an electromagnet or, in typical embodiments, a permanent magnet as above-described.

The first magnetic field source—and in embodiments having a plurality of magnetic field sources, the second and any additional magnetic field sources—are reversibly fixed to desired anatomic locations. In some embodiments, one or more magnetic field sources are reversibly fixed prior to surgery. In other embodiments, at least one magnetic field source is reversibly attached during surgery. In some embodiments, at least one magnetic field source is reversibly fixed to a desired anatomic location prior to surgery, and at least one during surgery.

One or more of the anatomic locations that is reversibly marked with a magnetic field source can usefully be outside the sterile surgical field. In such embodiments, the magnetic field source need not be sterile. In various embodiments, at least one of the anatomic locations reversibly marked with a magnetic field source is within the sterile surgical field. In such embodiments, the magnetic field source will be sterile prior to application. For maximum flexibility in usage, magnetic field sources for use in the present invention may be sterile, whether or not later used within or outside the sterile surgical field. Sterile magnetic field sources will typically be packaged within packaging that maintains sterility prior to surgery.

In one series of embodiments, at least one magnetic field source—in some embodiments, a plurality of magnetic field sources—are reversibly fixed to the patient's skin, permitting noninvasive determination of the position of the anatomic locations so marked. Reversible fixation to skin, e.g. unbroken skin, is typical of embodiments of the methods of the present invention in which the source is reversibly fixed outside the sterile surgical field.

For application to skin, the magnetic field source used in the methods of the present invention may usefully include an adhesive layer; in such embodiments, the magnetic field source is typically adhesively attached to the patient's skin.

Typically, the adhesive will be a pressure-sensitive, hypoallergenic adhesive. Such adhesives, compatible with reversible application to skin, are well known in the surgical and medical arts.

The adhesive layer can be sized identically to the skin-proximal surface of the magnetic field source, so as to neither overhang nor underhang the magnetic field source. In other embodiments, the adhesive layer may either underhang or more typically, overhang the skin-proximal surface of the magnetic field source. In certain embodiments, the magnetic field source may be positioned approximately in the center of an adhesive layer that overhangs the magnetic field source in a plurality, occasionally all, directions; the larger size of the adhesive layer, in certain of these embodiments, facilitates manual placement on the patient's skin.

In typical adhesive embodiments, the adhesive layer is itself overlaid, prior to use, with a removable backing paper.

In alternative embodiments of the methods of the present invention, the magnetic field source lacks an adhesive backing and is manually affixed prior to or during surgery using clinical grade reversible adhesive tape or strips, such as 3M™ Steri-Strip™ Adhesive Skin Closures (3M, Minneapolis, Minn.).

The first magnetic field source—and in embodiments in which a plurality of magnetic field sources are used, the second and any additional magnetic field sources—are reversibly fixed to desired anatomic locations.

For example, in total hip arthroplasty, a first magnetic field source may usefully be reversibly fixed to the patient's skin overlying (that is, external to) a point superior to the hip joint, and a second magnetic field source reversibly fixed to the patient's skin overlying a point inferior to the hip joint.

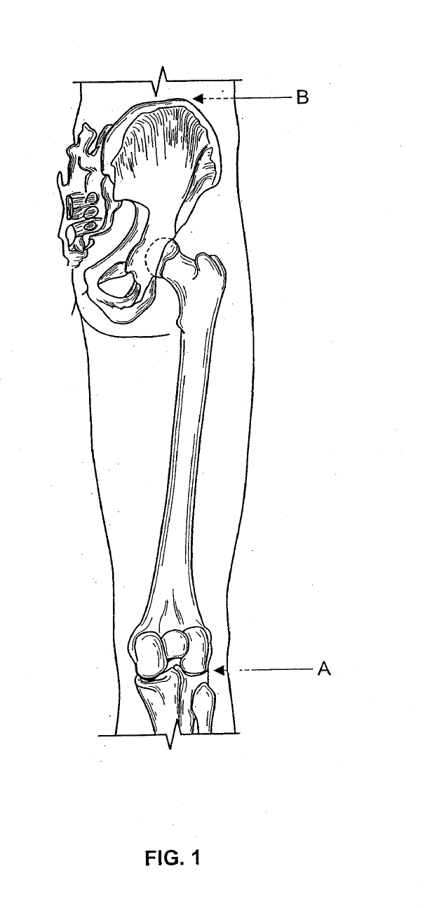

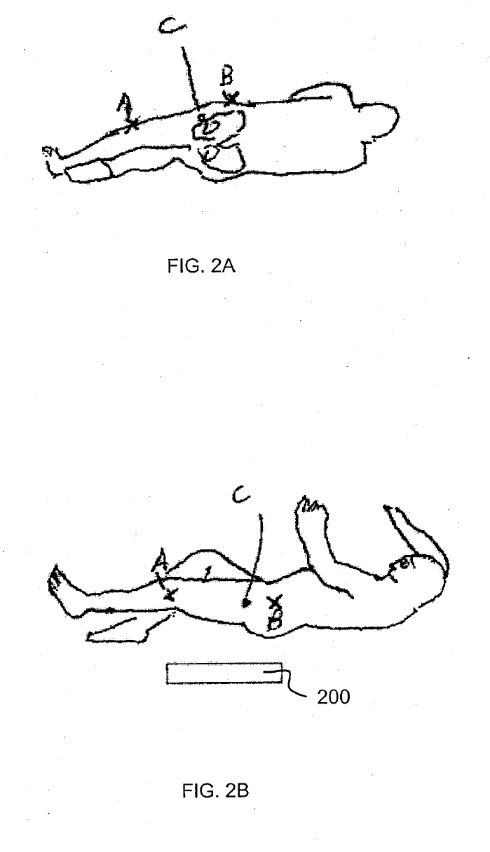

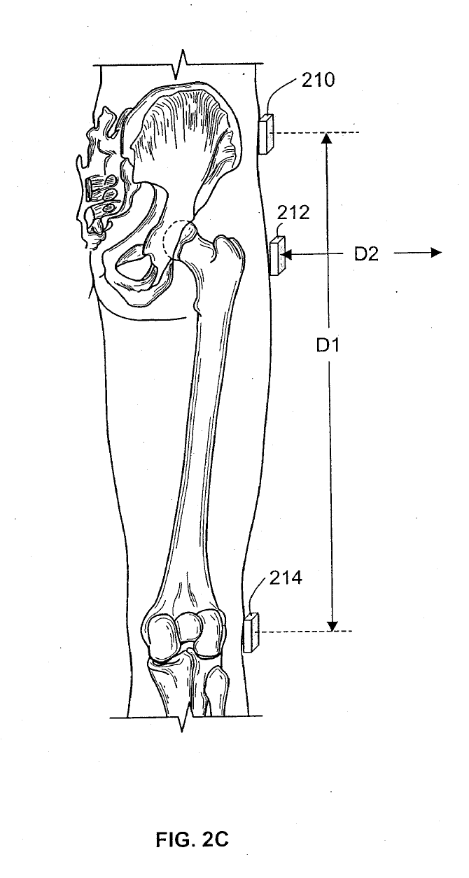

In some embodiments, for example, a first magnetic field source is reversibly fixed to the skin overlying the lateral iliac crest on the operative side, which is readily and reproducibly palpable and outside the sterile surgical field. This location is marked as “B” in FIGS. 1, 2A and 2B.

In certain embodiments, a second magnetic field source is usefully reversibly fixed to the patient's skin overlying the fibular head on the operative side, also readily palpable and outside the sterile surgical field. This location is marked “A” in FIGS. 1, 2A, and 2B.

In some embodiments, a third magnetic field source may be reversibly fixed on the lateral malleolus of the affected (operative) limb.

In total knee replacement surgery, as another example, a first magnetic field source may usefully be reversibly fixed to the patient's skin superior to the knee joint, and a second magnetic field source reversibly fixed to the patient's skin overlying a point inferior to the knee joint.

In some embodiments, the first anatomic location is ipsilateral to the second anatomic location, such as in embodiments in which a first marker is reversibly fixed to skin overlying the lateral iliac crest and a second marker is reversibly fixed to skin overlying the lateral fibular head, both on a patient's affected side. In other embodiments, the first anatomic location marked with a magnetic field source is contralateral to the second anatomic location, as, for example, on contralateral sides of a single limb.

In some embodiments, the methods of the present invention further comprise determining the distance between first and second anatomic locations, the first and second anatomic locations respectively marked by first and second magnetic field sources reversibly fixed thereto.

The distance may be determined by the magnetic field sensor device itself, or by the sensor device in conjunction with computational means, such as a digital computer, with which it is in communication. In some embodiments, the distance measurement reported by the sensor device (alone or in conjunction with computational means communicably attached thereto) is the absolute distance between first and second markers. In other embodiments, the distance measurement is reported relative to a reference distance; the reference distance, in some embodiments, is a preoperatively measured distance.

During total hip replacement surgery embodiments, the methods of the present invention may, therefore, usefully further comprise a determination of the distance between a first and a second magnetic field source, the first magnetic field source reversibly fixed to a first anatomic location that is superior to the hip joint, the second magnetic field source being reversibly fixed to a second anatomic location that is inferior to the affected hip joint, the distance so reported providing a measure of leg length. In some embodiments, the first magnetic field source is reversibly fixed to the skin overlying the lateral iliac crest, the second marker reversibly fixed to the skin overlying the lateral condyle of the tibial head, both on the lateral aspect of the affected hip, and the method further comprises a determination of the distance therebetween. This distance is illustrated as “D1” in FIG. 2C, the distance between magnetic field source 210, reversibly fixed to skin overlying the iliac crest, and magnetic field source 214, reversibly fixed to skin overlying the lateral condyle of the tibial head.

In certain embodiments of the methods of the present invention used during total hip replacement, a first measurement of leg length according to the methods of the present invention may usefully be made prior to first incision. A second measurement of leg length may usefully be made after insertion of trial acetabular and femoral prostheses and relocation of the prosthetic femoral head within the acetabular prosthesis. Such measurement usefully permits the surgeon to choose a prosthesis for permanent implantation that best equalizes the lengths of the affected and unaffected leg. Further measurements can be made post-operatively to confirm equalization of leg lengths and, optionally, to guide preparation of lifts or orthotics for patient use after surgery.

FIG. 2C further illustrates a second type of exemplary measurement that may usefully be made using the methods, sensor apparatus, kits and systems of the present invention.

With reference to FIG. 2C, magnetic field source 212 is reversibly fixed to skin overlying the greater trochanter of the femur, permitting measurement of the lateral position of the greater trochanter using the magnetic sensor apparatus of the present invention. The measurement, “D2”, may be made in absolute terms, and/or relative to the lateral position of other anatomic locations similarly marked, such as relative to the lateral position of the iliac crest, marked by reversible fixation of magnetic field source 210 to overlying skin.

Such measurements usefully permit changes in the hip offset to be measured and adjusted during THR surgery. Surgical increase in the lateral offset of the hip usefully increases the mechanical advantage of the abductor muscles and post-surgical hip stability.

In another aspect, the invention provides sensor apparatus adapted for intraoperative measurement of the spatial position of one or more anatomic points, each marked by at least one magnetic field source. The sensor apparatus comprises at least one sensor element capable of sensing and reporting the location and/or change in location of a magnetic field source.

Such sensor elements may vary in complexity.

For example, in certain embodiments, the sensor element may comprise a translucent sheet having particles of a ferromagnetic material suspended in the cells of the paper. Such a sensor element, available as “magnet paper” from Magnetic Component Engineering, Inc (Torrance, Calif.), can further comprise visible ruler markings. Bringing a sensing apparatus comprising at least one such sensing element into proximity with a twice-marked patient would permit the distance between first and second magnetic field sources to be read as the distance between darkened areas of the sensor element.

More typically, the sensor element is selected from the group consisting of variable reluctance, Hall effect, reed switch, and magnetoresistive sensors.

In some embodiments, the sensor apparatus comprises a plurality of sensor elements. For example, in some embodiments, the sensor apparatus comprises two sensor elements disposed at a fixed distance from one another; in other embodiments, the sensor apparatus comprises an array of sensor elements.

The sensing apparatus may usefully be shaped and dimensioned for hand use, either as a self-contained unit or, in alternative embodiments, as a hand-held unit tethered or tetherable to external devices.

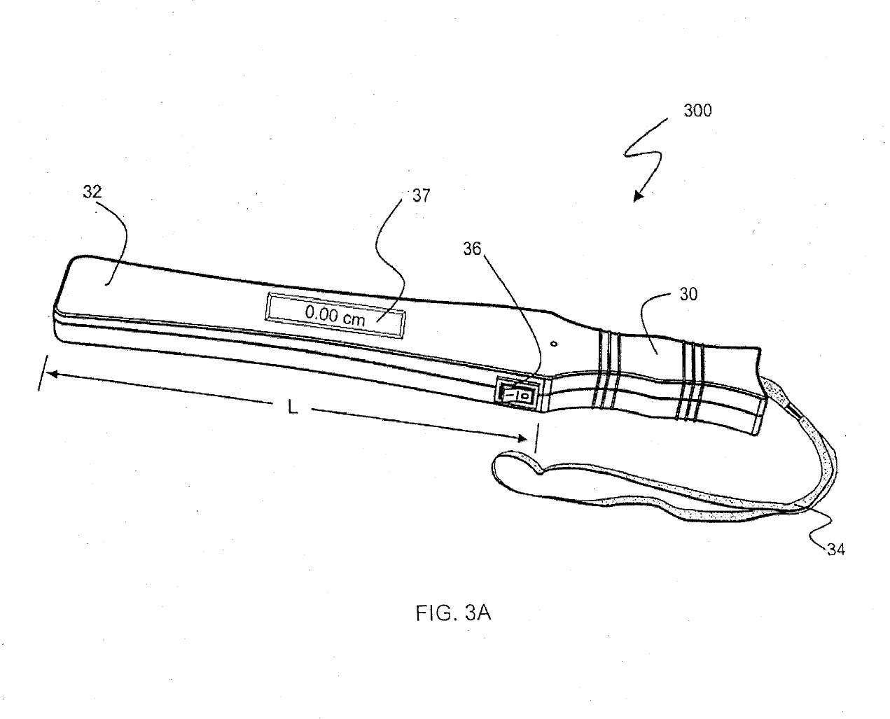

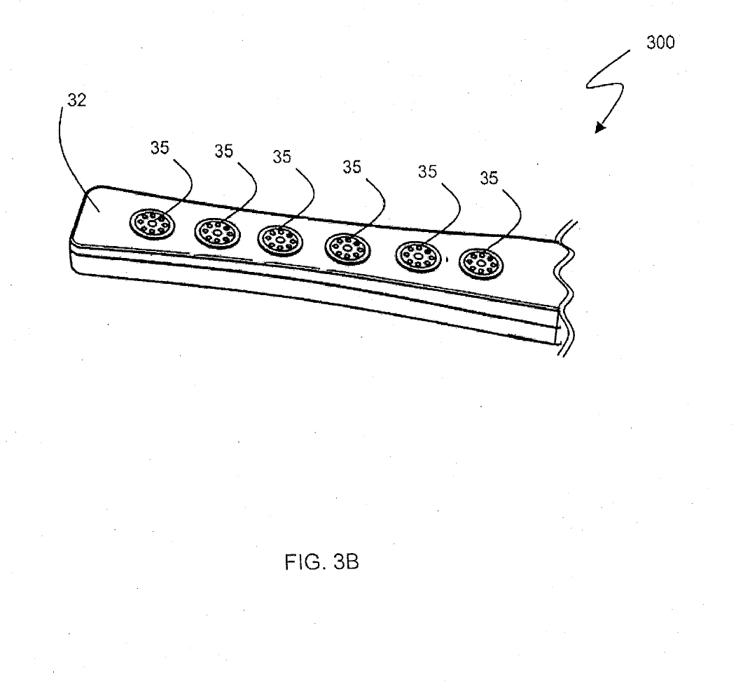

With reference to the exemplary sensor apparatus embodiment of FIG. 3A, self-contained handheld sensor apparatus 300 comprises handle 30, body 32, optional strap 34, and power switch 36. In the embodiment shown, power switch 36 is located on body 32; in other embodiments, power switch 36 is usefully located on handle 30. In yet other embodiments, sensor apparatus 300 lacks a power switch; in some of these embodiments, for example, apparatus 300 is constitutively powered “on” when removed from a docking device, not shown.

Body 32 has length “L” that, in typical embodiments, is at least as long as the distance to be measured between a first and a second magnetic field source. Thus, in various embodiments, body 32 is at least as long as 5 cm, 10 cm, 15 cm, even at least as long as 20 cm, 25 cm, 30 cm, 35 cm, 40 cm, 45 cm, even as long as 50 cm or more. In some embodiments, body 32 has length “L” at least as long as the distance between the superior portions of the lateral iliac crest and tibial head in 95%, 96%, 97%, even in 98% or 99% of adult patients.

Although device 300 is shown as being of integral manufacture, in some embodiments handle 30, with optional strap 34, is usefully detachable from body 32. These embodiments permit bodies 32 having different lengths “L” to be attached, depending upon the distance to be measured.

FIG. 3B is a partial bottom perspective view of apparatus 300, showing body 32. In the embodiment shown, body 32 comprises a plurality of magnetic sensor elements 35.

As discussed above, sensor elements 35 can be selected, for example, from the group consisting of variable reluctance, Hall effect, reed switch, and magnetoresistive sensors. Although a plurality of discrete sensors 35 are shown disposed substantially along the length “L” of body 32, other embodiments include a continuous array of sensors 35 (not shown), and yet other embodiments include two sensors, typically but not invariably disposed substantially at the handle-proximal and handle-distal ends of body 32.

Returning to the top perspective view of FIG. 3A, body 32 further comprises visual display means 37. Visual display means 37 can, for example, be a liquid crystal display. In other embodiments, visual display means 37 can be an LED display, active matrix display, TFT display, gas plasma display, or other types of visual displays well known in the computer arts. Display 37 is typically sized to permit reading from a distance, such as at arm's length distance.

Display 37 can, in some embodiments, display a numerical distance measured between a first and second magnetic field source. As discussed above, the distance may be an absolute distance or may be a distance relative to a reference distance. Display 37 may be adapted to display both types of measurement, with the actual data to be displayed determined by circuitry within apparatus 300, by computing means with which apparatus 300 is connected (either physically or via wireless communication), or both.

In some embodiments, display 37 is a color display, with positive measurements (as compared to a reference measurement) displayed in a first color, such as green, and negative measurements (as compared to a reference measurement) displayed in a second color, such as red. In other embodiments, the display is a monochrome display. In certain monochrome display embodiments (and some color display embodiments), positive and negative relative measurements are reported with a prefixed “+” or “−” sign, not shown.

Display 37 can, in alternative embodiments not shown, comprise ruler markings that change in color, luminance, or in other visually detectable property; in such embodiments, display 37 typically runs substantially along the length “L” of body 32.

Not shown in FIGS. 3A and 3B, self-contained handheld sensing apparatus 300 also comprises one or more internal batteries, either disposable batteries, such as alkaline AAA, AA, or 9V batteries, or rechargeable batteries, such as NiMH or NiCd or lithium rechargeable batteries. In some embodiments, the batteries are usefully disposed within handle 30; in other embodiments, the batteries are disposed with body 32; in yet other embodiments, the batteries are disposed within both handle 30 and body 32.

In certain embodiments, battery power is conserved between measurements by inclusion of an energy-conserving “sleep mode”, typically triggered automatically after a defined period, such as 1 minute, of nonuse. In such embodiments, body 32 usefully further comprises an optional wake-up button, not shown.

Also not shown, self-contained sensor apparatus 300 further comprises means required to process signals from the magnetic sensing elements, including, as required, analog-to-digital converters; and digital processing means to store, manipulate, compare and display such processed data, including both hardware and, as required, software executable thereon.

In various exemplary embodiments, apparatus 300 is held statically in proximity to, or is moved past, first and second magnetic field sources reversibly fixed to first and second anatomic locations on a surgical patient, typically but not invariably prior to first incision. Usefully, body 32 of apparatus 300 is so positioned as to be statically positioned or passed concurrently in proximity to first and second anatomic locations.

Apparatus 300 detects the location of magnetic field sources, either spontaneously or upon activation of a manual trigger (not shown). In some embodiments, the distance between first and second magnetic field sources is held in memory as a reference against which a subsequent measurement is compared. In other embodiments, the first measurement is displayed on display 37. Subsequent measurements are obtained similarly, with the distance between first and second magnetic field sources displayed either in absolute terms or relative to an earlier-obtained reference. In THR, the subsequent measurements are usefully made after implantation of trial femoral and acetabular prostheses.

As would be understood, the handheld self-contained embodiments of apparatus 300 usefully include means that permit the user to reset the stored memory values.

In some embodiments, hand held sensor apparatus 300 is physically connected, or tethered, to external devices. Such physical connection can, for example, usefully be made to handle 30 at the position indicated in FIG. 3A for insertion of optional strap 34. The physical connection can, for example, be used to deliver power to handle 30 and body 32, and can, in various embodiments, carry data communication lines for transmitting data to and receiving data from apparatus 300.

In various embodiments, apparatus 300 includes means for wirelessly communicating data to external devices. In some embodiments, apparatus 300 includes ports suitable for communicating data to reversibly attachable external devices.

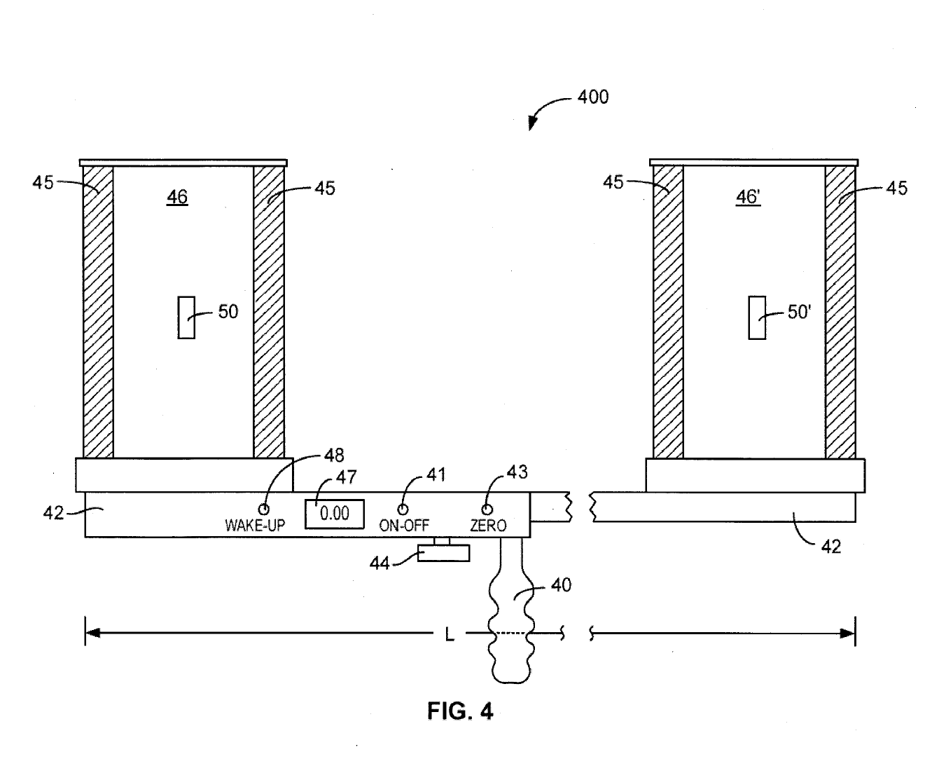

FIG. 4 provides a plan view of another exemplary embodiment of a hand held magnetic sensor for intraoperative use according to the present invention.

Apparatus 400 comprises body 42, optionally capable of extension, as by telescopic extension, along its long axis. Embodiments capable of extension usefully include lock 44 for fixing the initial length of body 42.

In typical embodiments, body 42 is capable of extension to a length at least as long as the distance to be measured between a first and a second magnetic field source. Thus, in various embodiments, body 42 is capable of extension to a length of at least 5 cm, 10 cm, 15 cm, even at least 20 cm, 25 cm, 30 cm, 35 cm, 40 cm, 45 cm, even as long as 50 cm or more. In some embodiments, body 42 is capable of extension to a length at least as long as the distance between the superior portions of the lateral iliac crest and tibial head in 95%, 96%, 97%, even in 98% or 99% of adult patients.

The initial length is usefully set at the outset of surgery to center first and second magnetic field sources 50 and 50′ within first and second windows 46 and 46′, respectively, as further described below.

Handle 40 attaches, fixedly or reversibly, to body 42. Reversible attachment of handle 40 usefully permits handles 40 having different dimensions to be used interchangeably, permitting apparatus 400 to be sized to an individual surgeon's grip. In other embodiments, fixed attachment of handle 40 to body 42 may be chosen for ease of, or to reduce cost of, manufacture.

Handle 42 can usefully be manufactured so as to facilitate grip, either by appropriate shaping, as for example by provision of outwardly extending ribs or protrusions, or by composition, as for example by provision of an inwardly compliant external surface, or both.

In the exemplary embodiment shown, body 42 comprises power switch 41. Power switch 41 may, in alternative embodiments, be located on handle 40, and in other embodiments may be omitted entirely.

Body 42 also comprises display 47.

In some embodiments, display 47 is a digital display, suitable for displaying numerical distance measurements, for example in centimeters, in 10 mm or 1 mm increments. In other embodiments, display 47 is suitable for displaying numerical distance measurements in mm or other units.

In some embodiments, the display is a color display, with positive measurements (as compared to a reference measurement) displayed in a first color, such as green, and negative measurements (as compared to a reference measurement) displayed in a second color, such as red. In other embodiments, the display is a monochrome display. In certain monochrome (and some embodiments of color) display embodiments, positive and negative relative measurements are reported with a prefixed “+” or “−” sign, not shown.

Display 47 can, in alternative embodiments not shown, comprise ruler markings that change in color, in luminance, or in other visually detectable property; in such embodiments, display 47 typically runs substantially along the length “L” of body 42.

In the embodiment exemplified in FIG. 4, body 42 further comprises zeroing button 43, which permits a first distance measurement to be obtained and the device set thereafter to calculate and display differential measurements therefrom. In other embodiments, zeroing button 43 is omitted, and device 400 reports absolute distance measurements. In yet other embodiments, device 400 can report either absolute or relative distance measurements. As would be understood, device 400 usefully (but does not invariably) further include means that permit the user to reset the stored memory values.

Not shown, self-contained handheld magnetic sensing device 400 contains one or a plurality of batteries, either disposable batteries, such as AAA, AA, 9V alkaline batteries, or rechargeable batteries, such as NiCd, NiMH, or lithium rechargeable batteries. In some embodiments, batteries are disposed within handle 40. In other embodiments, batteries are disposed within body 42. In other embodiments, batteries are disposed within both handle 40 and body 42.

In certain embodiments, battery power is conserved between measurements by inclusion of an energy-conserving “sleep mode”, typically triggered automatically after a defined period, such as 1 minute, of nonuse. In such embodiments, body 42 usefully further comprises optional wake-up button 48, as shown.

Also not shown in FIG. 4, self-contained sensor apparatus 400 further comprises means required to process signals from the magnetic sensing elements, including, as required, analog-to-digital converters; and digital processing means to store, manipulate, compare and display such processed data, including both hardware and, as required, software executable thereon.

In the exemplary embodiment shown in FIG. 4, sensor elements 45 extend outwardly from body 42, away from handle 40, to provide two sensing locations, each having a window (46 and 46′) through which first and second magnetic field sources reversibly fixed on a patient (50 and 50′) can respectively be visualized.

In exemplary uses during THR, a patient is positioned in the lateral decubitus position with affected hip elevated. A first magnetic field source is adhesively fixed to the skin overlying the lateral iliac crest on the affected side, and a second magnetic field source is adhesively fixed to the skin overlying the head of the tibia on the ipsilateral side. The patient is draped, and each magnetic field source, in turn, is palpated and its approximate position marked externally on the drape.

Prior to first incision, sensing apparatus 400 is positioned (in certain exemplary embodiments) over the patient and the length “L” of body 42 adjusted, and locked, so as to permit first magnetic field source 50 and second magnetic field source 50′ (or first and second marks on the surgical drapes that respectively overlie the field sources) to be viewed through respective windows 46 and 46′ of apparatus 400; typically, length “L” is adjusted so as approximately to center source 50 and 50′ within their respective viewing windows.

With apparatus 400 so positioned over the two magnetic field sources, zero button 43 is actuated to establish a preoperative distance, which is recorded. In some embodiments, recording the preoperative distance sets display 47 to zero.

As needed during surgery, apparatus 400 is positioned to detect the distance between first and second magnetic field sources, with the difference from the first-measured distance indicated on display 47. For example, during THR, measurement may usefully be made after implantation of a trial hip prosthesis, so as to facilitate the choice of prosthesis that best equalizes the length of the involved and uninvolved legs.

In some embodiments, hand held sensor apparatus 400 is physically connected, or tethered, to external devices. Such physical connection can, for example, usefully be made to handle 40, distal to its connection to body 42. The physical connection can, for example, be used to deliver power to handle 40 and body 42, and can, in various embodiments, carry data communication lines for transmitting data to and receiving data from apparatus 400.

In various embodiments, apparatus 400 includes means for wirelessly communicating data to external devices. In some embodiments, apparatus 400 includes ports suitable for communicating data to reversibly attachable external devices.

In other embodiments, the magnetic sensor apparatus of the present invention is not sized or dimensioned for handheld use.

For example, in an exemplary embodiment schematized in FIG. 2B, a top side view of a patient in lateral decubitus position, apparatus 200 is fixedly positioned behind the patient. In certain of these embodiments, apparatus 200 usefully takes measurements without user intervention, either continuously or periodically. In other embodiments, apparatus 200 can be triggered to take measurements through use of an actuating device, such as a button on apparatus 200, a button operably attached to apparatus 200, including, for example, a foot-operated actuator.

In yet other embodiments, the sensor apparatus of the present invention lacks one or more of means required to process signals from the magnetic sensing elements; digital processing means to store, manipulate, compare and display such processed data, including either or both of hardware and software executable thereon; and a display.

In such embodiments, the means required to process signals, the digital processing means, and/or display are conveniently located on an external device with which the sensor apparatus is communicably connected. Communication can be wireless (as, e.g., by BlueTooth, WiFi, dedicated RF link) or wired.

Whether hand-held or not, fully self-contained or not, in typical embodiments the magnetic sensor apparatus of the present invention is so configured as to permit a plurality of magnetic sensor elements to be concurrently positioned in operable proximity to a plurality of magnetic field sources disposed ipsilaterally on a surgical patient. In embodiments having a rigid body, such as those above-described, this is typically effected by disposing a plurality of magnetic sensor elements on a common face of the sensor apparatus body. In embodiments having a malleable or articulated body, the plurality of sensor elements may be disposed on the body so as to be so positionable after bending, twisting, or other deformation of the body of the apparatus.

In another aspect, the invention provides kits comprising a plurality of magnetic field sources adapted for reversible fixation to desired anatomic locations of surgical patients.

Typically, each of the plurality of magnetic field sources is a permanent magnet. The magnets in such kits can be any of the magnets above-described for use in the methods of the present invention. For example, in certain kit embodiments, each of the plurality of magnets possesses an adhesive backing.

Kits may comprise two, three, four, or more magnetic field sources, such as permanent magnets. In some embodiments, each of the plurality of magnets included within the kit is separately packaged. In some embodiments, each of the plurality of magnets is separately packaged to maintain sterility.

In various embodiments, all of the plurality of magnets are sterile; in other embodiments, the magnets are clean but not sterile.

In some embodiments, all of the plurality of magnets included in the kit have approximately the same field strength. In some embodiments, all of the plurality of magnets included in the kit have approximately the same size and shape. In some embodiments, all of the plurality of magnets included in the kit have magnetization axis and polarity oriented identically with respect to the magnet's shape.

In some embodiments, at least one of the plurality of magnets is dissimilar from others of the plurality in any one or more of field strength, size, shape, magnetization axis, and polarity. In such embodiments, the one or more dissimilar magnets will typically be visually distinguishable, as by size, color, shape, or packaging. Use of magnets differing in one or more of the above-described parameters facilitates position and/or distance measurements using certain embodiments of the magnetic sensor apparatus of the present invention.

In some kit embodiments, the kit further comprises adhesive tape or strip suitable for reversible fixation of the magnetic field source, such as permanent magnet, to the patient's skin. The kit may also comprise means for prepping patient skin to facilitate adherence of the magnetic field source, including, for example, alcohol and/or betadine swabs or pads.

In another aspect, the invention provides a system for determining the distance between at least a first and second anatomic location of a patient during surgery. The system comprises at least a first and second magnetic field sources, each of the magnetic field source reversibly fixable to a desired anatomic location of a surgical patient; and a sensor apparatus, the sensor apparatus capable of measuring the distance between first and second magnetic field sources.

In typical embodiments, the magnetic field sources are permanent magnets, including any of the embodiments above-described. In various embodiments, the sensor apparatus is a sensor apparatus of the present invention, including any of the embodiments above-described.

All references cited throughout the specification and the references cited respectively therein are hereby expressly incorporated by reference herein.

While the foregoing invention has been described in some detail by way of illustration and example, it would be understood by those skilled in the art that various changes may be made, equivalents substituted, and embodiments combined without departing from the true spirit and scope of the invention. All such modifications and equivalents are within the scope of the present invention as defined by the following claims and their equivalents.

What is claimed:

1. A method for determining the spatial position of at least one anatomic location of a patient during surgery, the method comprising:
intraoperatively detecting the spatial position of a first magnetic field source reversibly fixed to a first desired anatomic location of a patient.

2. The method of claim 1, further comprising detecting the spatial position of a second magnetic field source fixedly attached to a second desired anatomic location of a patient.

3. The method of claim 2, further comprising determining the distance between said first and second magnetic field sources.

4. The method of any one of claims 1, wherein said first and/or second magnetic field source is a permanent magnet.

5. The method of any one of claims 1, wherein said first and/or second magnetic field source is reversibly fixed outside the sterile surgical field.

6. The method of claim 5, wherein said first and/or second magnetic field source is reversibly fixed to the patient's unbroken skin.

7. The method of claim 6, wherein said first and/or second magnetic field source is adhesively attached to the patient's skin.

8. The method of any one of claims 2, wherein said first anatomic location is skin overlying a point superior to the hip joint, and said second anatomic location is skin overlying a point inferior to the hip joint.

9. The method of claim 8, wherein said first anatomic location is skin overlying the lateral iliac crest.

10. The method of claim 9, wherein said second anatomic location is skin laterally overlying the tibial head.

11. The method of any one of claims 1, wherein said surgery is total hip replacement surgery.

12. The method of any one of claims 1, wherein said first anatomic location is ipsilateral to said second anatomic location.

13. The method of claim 3, further comprising the antecedent step of:
fixedly attaching said first and second magnetic field sources respectively to said first and second desired anatomic locations.

14. The method of claim 13, further comprising the step, after fixedly attaching said magnetic field sources, of performing a preoperative determination of the distance between said first and second magnetic field sources.

15. The method of claim 11, further comprising the subsequent step of:
choosing a femoral and/or acetabular prosthesis for implantation based upon the distance measured between said first and second magnetic field sources.

16. A sensor apparatus for measuring the distance between first and second anatomic locations of a patient during surgery, the anatomic locations respectively marked with first and second magnetic field sources, the apparatus comprising:
a body;
at least first and second magnetic sensing elements, said first and second sensing elements so disposed on said body as to permit their concurrent approximation to first and second magnetic field sources disposed ipsilaterally on a surgical patient; and
means for determining the distance between said concurrently sensed first and second magnetic field sources.

17. The sensor apparatus of claim 16, wherein said at least first and second sensor elements are disposed on the same face of a rigid body.

18. The sensor apparatus of claim 16, wherein said body is so dimensioned as to permit said first and second sensing elements to be concurrently approximated to first and second magnetic field sources disposed on skin that is respectively external to the lateral iliac crest and tibial head of an adult patient.

19. The sensor apparatus of any one of claims 16, wherein said distance determining means is capable of determining an absolute distance between the first and second magnetic field sources.

20. The sensor apparatus of any one of claims 16, wherein said distance determining means is capable of determining a change in distance between first and second magnetic field sources as between successive measurements.

21. The sensor apparatus of claim 20, further comprising means for setting a first measured distance between first and second magnetic field sources as a reference for determining a change in distance therebetween in successive measurements.

22. The sensor apparatus of any one of claims 16, further comprising a visual display, the visual display disposed on the body in operable communication with the distance determining means.

23. The sensor apparatus of claim 22, wherein said display is configured to display distances numerically.

24. The sensor apparatus of any one of claims 16, further comprising a handle, the apparatus dimensioned so as to permit hand-held use.

25. The sensor apparatus of claim 24, further comprising at least one battery, the at least one battery disposed internal to said body or said handle.

26. A kit for facilitating the measurement of the spatial distance between first and second anatomic locations of a patient during surgery, the kit comprising:
a plurality of magnetic field sources, at least a first of said plurality being suitable for reversible fixation to a first anatomic location of a surgery patient and at least a second of said plurality being suitable for reversible fixation to a second anatomic location of a surgery patient.

27. The kit of claim 26, wherein each of said plurality of magnetic field sources is a permanent magnet.

28. The kit of claim 27, wherein said magnets further comprise an adhesive layer, the adhesive compatible with reversible fixation to human skin.

29. The kit of claim 28, wherein the adhesive layer is overlaid on the skin-proximal side of the magnet with a removable backing layer.

30. The kit of claim 26, wherein each of said plurality of magnets is sterile.

31. The kit of claim 30, wherein each of said sterile magnets is separately packaged.

32. A system for determining the distance between at least a first and second anatomic location of a patient during surgery, comprising:
at least a first and second magnetic field source, each said magnetic field source reversibly fixable to a desired anatomic location of a surgical patient; and
a sensor apparatus, the sensor apparatus capable of measuring the distance between first and second magnetic field sources.

33. The system of claim 32, wherein the first and second magnetic field sources are permanent magnets.

34. A system for determining the distance between at least a first and second anatomic location of a patient during surgery, comprising:
at least a first and second magnetic field source, each said magnetic field source reversibly fixable to a desired anatomic location of a surgical patient; and
a sensor apparatus, the sensor apparatus capable of measuring the distance between first and second magnetic field sources, wherein the sensor is a sensor according to claim 16.
